(19) United States
(12) Patent Application Publication
(10) Pub. No.: US 2002/0107282 A1
(43) Pub. Date: Aug. 8, 2002

(54) Cosmetic composition containing an aminophenol derivative and an isoflavonoid

(75) Inventors: Veronique Chevalier, Villecresnes (FR); Dang-Man Pham, Sucy-En-Brie (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/986,885

(22) Filed: Nov. 13, 2001

(30) Foreign Application Priority Data
Nov. 10, 2000 (FR) 00 14479

(51) Int. Cl. A61K 31/7048; A61K 31/353; A61K 31/325; A61K 31/18; A61K 31/17

(52) U.S. Cl. 514/486; 514/546; 514/596; 514/605; 514/456; 514/27

(57) ABSTRACT

The invention relates to a composition containing at least one aminophenol derivative and at least one isoflavonoid. These combinations of active agents show synergism in the depigmentation and/or bleaching of the skin.

### BACKGROUND OF THE INVENTION

1. Field of the Invention

The present invention relates to a composition containing, preferably in a physiologically acceptable medium, at least one aminophenol derivative and at least one isoflavonoid. The invention also relates to the preparation and the use of this composition, for example in a cosmetic and/or dermatological manner for depigmenting and/or bleaching human skin.

2. Discussion of the Background

For several years, considerable effort has been devoted to proposing harmless topical depigmenting substances with good efficacy. The use of such substances with good efficacy is most particularly desired for treating regional hyperpigmentations caused by melanocyte hyperactivity, such as idiopathic melasmas, occurring during pregnancy (“pregnancy mask” or chloasma) or during oestro-progestative contraception, localized hyperpigmentations caused by hyperactivity and proliferation of benign melanocytes, such as senile pigmentation marks known as actinic lentigo, accidental hyperpigmentations or depigmentations, possibly due to light-induced sensitization or to post-lesional cicatrization, and also certain leukodermas, such as vitiligo. For these conditions (in which the cicatrizations can result in a scar which gives the skin a whiter appearance) and for leukodermas, failing the ability to repigment the damage to skin, the regions of residual normal skin are depigmented in order to give the skin an overall uniform white complexion.

Various depigmenting agents have thus been proposed. Among the most well known, mention may be made of: hydroquinone and its derivatives such as arbutin and its esters; ascorbic acid and its derivatives, especially glycosylated derivatives; kojic acid and its esters; ellagic acid and its derivatives; certain plant extracts, and especially extract of liquorice, of mulberry or of skullcap; and also glutathione, cystein and precursors thereof. Moreover, L'Oreal has demonstrated that certain aminophenol derivatives have the property of inhibiting melanogenesis even at low concentrations, without showing any cytotoxicity (WO 99/10318, incorporated herein by reference).

### SUMMARY OF THE INVENTION

The Inventors have now discovered, unexpectedly, that the combination of an aminophenol derivative with an isoflavonoid has depigmenting power and/or bleaching power. This power can be superior to that of these compounds when considered separately. In other words, the invention combination potentiates the efficacy of these compounds, which in a preferred embodiment produces a synergistic effect in bleaching and/or depigmenting the skin.

### DETAILED DESCRIPTION OF THE INVENTION

Isoflavonoids are a subclass of flavonoids, formed from a 3-phenylchroman skeleton which may comprise various substituents and different levels of oxidation. In contrast with flavonoids, they are present in only a very limited number of plants.

The term “isoflavonoid” includes several classes of compounds, among which mention may be made of isoflavones, isoflavanones, rotenoids, pterocarpans, isoflavans, isoflavan-3-enes, 3-arylcoumarins, 3-aryl-4-hydroxycoumarins, coumestans, coumaronochromones, α-methyldeoxybenzoins or 2-arylbenzofurans. In this regard, for a review of isoflavonoids, methods for analyzing them and their sources, reference may advantageously be made to Chapter 5 of “Isoflavonoids” written by P. M. Dewich, in The Flavonoids, published by Harbone, pp. 125-157 (1998), incorporated herein by reference. The isoflavonoids mentioned in this chapter are useful herein. While certain isoflavonoids are known as depigmenting agents, to the Inventor's knowledge, it has never yet been envisaged to combine them with aminophenol derivatives.

As noted above, one subject of the present invention is a composition comprising, consisting essentially of, consisting of, optionally in a physiologically acceptable medium, at least one aminophenol derivative and at least one isoflavonoid. In a preferred embodiment, the combination of isoflavonoid and aminophenol derivative provide a greater bleaching and/or depigmenting effect on human skin as compared to equal moles of the individual materials. Any statistically significant improvement is meant by “greater,” such as 1%, 3%, 5%, 10%, 20%, 50%, 150%, etc. Such combinations are referred to as “synergistic combinations.” Such synergistic combinations are easily determined by those of ordinary skill in the art in view of the teachings herein. As an example, an invention composition comprising isoflavonoid I and aminophenol derivative A would be an invention synergistic combination of the bleaching and/or depigmenting effect on human skin if the combination of A and I was statistically significantly greater than that of I individually and A individually on an equal mole basis.

The expression “physiologically acceptable medium” means a medium which is suitable for topical application to the skin or its integuments, that is to say which is compatible with the skin and the nails.

Another subject of the invention is the preparation of, and cosmetic use of, the invention compositions, for example as a depigmenting and/or bleaching preparation for the skin.

Another subject of the invention is the use of the invention composition to manufacture a depigmenting and/or bleaching preparation for human skin.

The present invention also relates to a process for depigmenting and/or bleaching human skin, which comprises, consists essentially, and consists of applying a composition according to the invention to the skin.

The aminophenol compounds according to the invention preferentially correspond to formula (I) below:

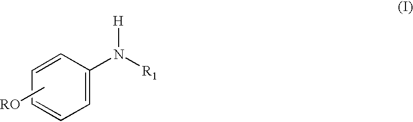

in which:

R represents a hydrogen atom or a radical —COR₂,

with R₂ representing a radical chosen from a saturated or unsaturated, linear, cyclic or branched C₁-C₃₀, optionally hydroxylated alkyl or alkoxyl radical,

R₁ is chosen from a radical of formula (a), (b) or (c) below:

(a) —CO—NR₃R₄

(b) —CO—O—R₅

(c) —SO₂—R₅

with R₃ representing a hydrogen atom or a linear or branched, saturated or unsaturated, optionally hydroxylated C₁-C₆ alkyl radical,

with R₄ representing a hydrogen atom or a radical chosen from a saturated or unsaturated, linear, branched or cyclic, optionally hydroxylated C₁-C₃₀ alkyl radical,

with R₅ representing a radical chosen from a saturated or unsaturated, linear, cyclic or branched, optionally hydroxylated C₁-C₃₀ alkyl radical,

as well as their addition salts with a (preferably physiologically acceptable) acid or base.

These compounds have the advantage of being easy to obtain. They may be especially obtained by reacting an aminophenol with an activated chemical species, such as an imidazolide or an isocyanate, when R₁ is a radical of formula (a), a chloroformate when R₁ is a radical of formula (b), or a sulphonyl chloride when R₁ is a radical of formula (c). Such is within the skill of the ordinary artisan.

Among the linear or branched alkyl radicals containing from 1 to 30 carbon atoms, those which may advantageously be mentioned are methyl, ethyl, propyl, isopropyl, butyl, tert-butyl, hexyl, octyl, nonyl, 2-ethylhexyl, dodecyl, tridecyl, hexadecyl, behenyl, octadecyl, tetracosyl, hexacosyl, octacosyl, myricyl, 2-butyloctyl and 2-hexyldecyl radicals. Even more preferentially, the alkyl radical generally contains from 1 to 6 carbon atoms and may be chosen from methyl, ethyl, propyl, isopropyl, tert-butyl and hexyl radicals.

When the alkyl radical is unsaturated, a radical containing one or more ethylenic unsaturations is preferred, such as neryl, 2-nonyl-2-butenyl, 6-(1,3-pentadienyl)-2,4,7-dodecanetrien-9-ynyl radicals and more particularly the allyl radical.

When the alkyl radical is cyclic, mention may especially be made of the cyclohexyl, cholesteryl or tert-butylcyclohexyl radical.

When it is hydroxylated, the radical preferably contains 1 to 6 carbon atoms and 1 to 5 hydroxyl groups.

Among the monohydroxyalkyl radicals, a preferred radical contains 1 to 3 carbon atoms, especially hydroxymethyl, 2-hydroxyethyl, or 2- or 3-hydroxypropyl radicals.

Among the polyhydroxyalkyl radicals, a radical that is preferred is a radical containing from 3 to 6 carbon atoms and from 2 to 5 hydroxyl groups, such as 2,3-dihydroxypropyl, 2,3,4-trihydroxybutyl, 2,3,4,5-tetrahydroxypentyl and 2,3,4,5,6-pentahydroxyhexyl radicals.

The alkoxylated radicals are alkyl radicals, especially such as described above, preceded by an oxygen atom.

Preferably, the aminophenol derivatives of formula (I) meet at least one and more preferably two or more, or all, of the conditions below:

R represents a hydrogen atom,

the function —OR on the phenyl radical is in an ortho position, or more preferably, in the para position, to the NHR₁, group,

R₁ is chosen from a radical of formula (a) or (b).

Preferred compounds of formula (I) according to the invention include N-ethyloxycarbonyl-4-aminophenol; N-ethyloxycarbonyl-O-ethyloxycarbonyl-4-aminophenol; N-cholesteryloxycarbonyl-4-aminophenol and N-ethylaminocarbonyl-4-aminophenol. In the context of the present invention, N-ethyloxycarbonyl-4-aminophenol is preferably used.

The isoflavonoids that are suitable for use in the present invention may be of natural or synthetic origin. The expression “natural origin” means the isoflavonoid in pure form or as a solution at various concentrations, obtained by various processes of extraction from an element, generally a plant, of natural origin. The expression “synthetic origin” means the isoflavonoid in pure form or as a solution at various concentrations, obtained by chemical synthesis.

Isoflavonoids of natural origin are preferably used. Included are: daidzin, genistin, daidzein, formononetin, cuneatin, genistein, isoprunetin and prunetin, cajanin, orobol, pratensein, santal, junipegenin A, glycitein, afrormosin, retusin, tectorigenin, irisolidone, and jamaicin, and also analogues and metabolites thereof, and mixtures thereof.

Isoflavones are preferred for use in the present invention. This term covers both the aglycone forms (e.g., daidzein, genistein and glycitein) and the glycosylated forms (e.g., daidzine, genistine, glycitine) of the isoflavones.

Processes for preparing isoflavones are described especially in WO 95/10530, WO 95/10512, U.S. Pat. No. 5,679,806, U.S. Pat. No. 5,554,519, EP-812 837 and WO 97/26269, all incorporated herein by reference. A preferred material is a soybean extract with a 37% titre of isoflavones that is sold by the company Maruzen under the name Fujiflavones P40.

As explained above the aminophenol derivative and the isoflavonoid are preferably present in the composition according to the invention in an amount such that they act synergistically to give the composition a depigmenting and/or bleaching effect which is superior to that obtained with a composition containing only one of these compounds. Mixtures may be used.

While not limited to particular amounts, the aminophenol derivatives may be present in the invention composition in an amount ranging from 0.01 to 10% by weight, preferably from 0.5% to 3% of the total weight of the composition, including 0.05, 1, 2, 4, 6, 8 and 9%.

For its part, the isoflavonoid, while not limited to particular amounts, preferably represents from 0.0001 to 10%, and more preferably from 0.001 to 1% of the total weight of the composition, including 0.01, 0.1, 0.3, 0.5, 0.7, 0.9, 2, 3, 4, 6, 8 and 9%. Mixtures maybe used.

Needless to say, if the isoflavonoid is present in the form of a solution containing a plant extract, a person skilled in the art will be able to adjust the amount of this solution in the composition according to the invention so as to obtain a desired concentration range, including the above concentration ranges of isoflavonoid.

The form of the composition of the invention is not limited. It may be, for example, in any pharmaceutical form normally used for topical application, especially in the form of an aqueous, aqueous-alcoholic or oily solution, an oil-in-water or water-in-oil or multiple emulsion, an aqueous or oily gel, a liquid, pasty or solid anhydrous product, a dispersion of oil in an aqueous phase with the aid of spherules, these spherules possibly being polymer nanoparticles such as nanospheres and nanocapsules or, better still, lipid vesicles of ionic and/or nonionic type.

The invention composition can be more or less fluid and can have the appearance of a white or coloured cream, an ointment, a milk, a lotion, a serum, a paste or a mousse. It may optionally be applied to the skin in aerosol form. It may also be in solid form and, for example, in the form of a stick. It may be used as a care product and/or as a make-up product for the skin.

The composition according to the invention can also contain adjuvants known in the cosmetics fields, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the field under consideration, and, for example, from 0.01 to 20% of the total weight of the composition. Depending on their nature, these adjuvants can be introduced for example into the fatty phase, into the aqueous phase, into lipid vesicles and/or into nanoparticles. In any case, these adjuvants and the proportions thereof will be chosen so as not to harm the desired properties of the combination of agents according to the invention.

When the composition according to the invention is an emulsion, the proportion of the fatty phase can range from 5% to 80% by weight and preferably from 5% to 50% by weight relative to the total weight of the composition. The oils, emulsifiers and coemulsifiers used in the composition in emulsion form can be chosen from those used conventionally in the field considered. The emulsifier and the co-emulsifier may be present in the composition in a proportion ranging from 0.3% to 30% by weight and preferably from 0.5% to 20% by weight relative to the total weight of the composition.

As oils which can be used in the invention, while not limited, mention may be made of mineral oils (liquid petroleum jelly) and esters thereof, oils of plant origin (avocado oil, soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax, ozokerite) can also be used as fatty substances.

As emulsifiers and coemulsifiers which can be used in the invention, although not limited mention may be made, for example, of fatty acid esters of polyethylene glycol such as PEG-20 stearate, and fatty acid esters of glycerol such as glyceryl stearate.

As hydrophilic gelling agents, while not limited, mention may be made in particular of carboxyvinyl polymers (carbomer), acrylic copolymers such as acrylate/alkylacrylate copolymers, polyacrylamides, polysaccharides, natural gums and clays, and, as lipophilic gelling agents, mention may be made of modified clays such as bentones, metal salts of fatty acids, hydrophobic silica and polyethylenes.

As active agents, the composition may preferably comprise at least one UV screening agent and/or one keratolytic agent and/or desquamating agent.

UV screening agents which may be used according to the invention include for example, and in a non-limiting manner, the following families (the names correspond to the CTFA nomenclature of the screening agents):

anthranilates, in particular menthyl anthranilate; benzophenones, in particular benzophenone-1, benzophenone-3, benzophenone-5, benzophenone-6, benzophenone-8, benzophenone-9, benzophenone-12 and, preferentially, benzophenone-2 (oxybenzone), or benzophenone-4 (Uvinul MS40 available from BASF); benzylidenecamphors, in particular 3-benzylidenecamphor, benzylidenecamphorsulphonic acid, camphor benzalkonium methosulphate, polyacrylamidomethylbenzylidenecamphor, terephthalylidenedicamphorsulphonic acid and, preferentially, 4-methylbenzylidenecamphor (Eusolex 6300 available from Merck); benzimidazoles, in particular benzimidazilate (Neo Heliopan AP available from Haarmann and Reimer), or phenylbenzimidazolesulphonic acid (Eusolex 232 available from Merck); benzotriazoles, in particular drometrizoletrisiloxane, or methylenebis-benzotriazolyltetramethylbutylphenol (Tinosorb M available from Ciba); cinnamates, in particular cinnoxate, DEA methoxycinnamate, diisopropyl methylcinnamate, glyceryl ethylhexanoate dimethoxycinnamate, isopropyl methoxycinnamate, isoamyl cinnamate and, preferentially, ethocrylene (Uvinul N35 available from BASF), ethylhexyl methoxycinnamate (Parsol MCX available from Hoffmann La Roche) or octocrylene (Uvinul 539 available from BASF); dibenzoylmethanes, in particular butylmethoxydibenzoylmethane (Parsol 1789); imidazolines, in particular ethylhexyldimethoxy-benzylidenedioxoimidazoline; PABAs, in particular ethyldihydroxypropyl PABA, ethylhexyldimethyl PABA, glyceryl PABA, PABA, PEG-25 PABA and, preferentially, diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate, or TEA salicylate; triazines, in particular anisotriazine (Tinosorb S available from Ciba).

The invention will now be illustrated with the aid of the non-limiting examples which follow.

### EXAMPLE 1

### Demonstration of the Synergistic Effect of the Compounds According to the Invention

An in vitro biological test (inhibition of the activity of fungal tyrosinase) demonstrated the depigmenting acitivity of the combinations of compounds according to the invention.

This test consists in measuring the absorbence (optical density OD) at 475 nm as a function of time for each of the depigmenting active agents tested.

These agents are dissolved (at various concentrations) in a suitable solvent in the presence of a phosphate buffer, 1 ml of L-tyrosine at 2.0×10⁻³ M in a phosphate buffer, 0.1 ml of L-Dopa at 1.0×10⁻⁴ M in a phosphate buffer and 50 μl of a fungal tyrosinase solution at 1 g/l in a phosphate buffer, compared with a control consisting of the solvent used at the same concentration in the phosphate buffer. A percentage of inhibition is then determined by the following equation:

$$\frac{{DO}_{max , 475 nm ( control )} - {DO}_{max , 475 nm ( activeagent )}}{{DO}_{max , 475 nm ( control )}} \times 100$$

For each of the active compounds studied, the molar concentration for which an inhibition value of close to 25% should be observed (theoretical IC) is then determined. The concentrations C₁ and C₂ corresponding to each active agent used alone are thus obtained. The inhibition value actually obtained at the concentrations C₁ and C₂ is then checked (IC obtained). These two active agents in the concentrations C₁ and C₂ found above are then mixed together. The theoretical percentage of inhibition of the mixture (or theoretical IC), corresponding to the sum of the percentages of inhibition observed for the individual compounds, is then calculated, and the percentage of inhibition obtained (or measured IC) is measured under the same experimental conditions as for the compounds tested separately.

By way of example, the results obtained with N-ethyloxycarbonyl-4-aminophenol and a soybean extract with a 37% titre of isoflavones (sold by the company Maruzen under the name Fujiflavone P40) are as follows:

| | | Theoretical | IC obtained |
|---|---|---|---|
| Active agents | Concentration C | IC (%) | (%) |
| N-ethoxy-carbonyl-4- | C₁ = 1.25 × 10⁻⁴ | 24.8 | 23.5 |
| aminophenol/ethanol | M | | |
| Soybean extract | C₂ = 0.04% | 24.0 | 31.4 |
| (Fujiflavone P40)/DMSO | | | |

| | Expected theoretical | Measured |
|---|---|---|
| Combination of active agents | IC (%) | IC (%) |
| N-ethoxy-carbonyl-4-aminophenol | 54.9 | 72.6 |
| and soybean extract | | |

It emerges clearly that the measured IC is markedly greater than the theoretical IC for the mixture of active agents.

Given the precision of this test (of the order of 5%), the synergistic effect of the two compounds in combination is thus clearly demonstrated, and an example of a synergistic combination is provided.

### EXAMPLE 2

### Oleosome-Type O/W Fluid

The following composition is prepared.
The amounts are indicated as percentages by weight.

| Phase A | | |
|---|---|---|
| N-ethyloxycarbonyl-4-aminophenol | | 0.5% |
| Stearyl heptanoate and stearyl octanoate | | 5.5% |
| Plant oils | | 11.6% |
| UVA screening agent | | 1.9% |
| Cyclopentasiloxane | | 3.7% |
| Sucrose tristearate | | 2.0% |
| Oxyethylenated (4 EO) sorbitan monostearate | | 1.35% |
| i Stearic acid | | 1.0% |
| Oxyethylenated (60 EO) hydrogenated castor oil | | 2.5% |
| Tocopheryl acetate | | 0.5% |
| Phase B | | |
| Fujiflavone P40 (Maruzen) | | 0.1% |
| Preserving agents | | 0.29% |
| Triethanolamine | | 0.50% |
| Disodium EDTA | | 0.05% |
| Glycerol | | 3.0% |
| Water | qs | 100.0% |

A bleaching fluid for the face is obtained.

The fluid may be obtained in the following manner: Phases A and B are separately heated to 65° C. and then cooled to room temperature. Phase B is then introduced into phase A in order to obtain a W/O emulsion which is then homogenized in a high-pressure homogenizer (500 b, 1 to 3 treatments).

### EXAMPLE 3

### Oleosome-Type O/W Fluid

| Phase A | | |
|---|---|---|
| N-ethyloxycarbonyl-4-aminophenol | | 0.5% |
| Stearyl heptanoate and stearyl octanoate | | 5.5% |
| Plant oils | | 11.6% |
| UVA screening agent | | 1.9% |
| Cyclopentasiloxane | | 3.7% |
| Sucrose tristearate | | 2.0% |
| Oxyethylenated (4 EO) sorbitan monostearate | | 1.35% |
| Stearic acid | | 1.0% |
| Oxyethylenated (60 EO) hydrogenated castor oil | | 2.5% |
| Tocopheryl acetate | | 0.5% |
| Phase B | | |
| Flavosterone SB (Ichimaru Pharcos) | | 10.0% |
| Preserving agents | | 0.29% |
| Triethanolamine | | 0.50% |
| Disodium EDTA | | 0.05% |
| Glycerol | | 3.0% |
| Water | qs | 100.0% |

This bleaching fluid for the face may be obtained according to the same process as in Example 2.

### EXAMPLE 4

### Depigmenting Gel

| Phase A | | |
|---|---|---|
| UVB screening agent | | 0.5% |
| Cyclohexasiloxane | | 5.0% |
| Phase B | | |
| Soy Life 150 NGMO (Schouton Products) | | 0.5% |
| Preserving agents | | 0.65% |
| Disodium EDTA | | 0.1% |
| Sodium hydroxide | | 0.11% |
| Glycerol | | 10.0% |
| Ethanol | | 2.5% |
| N-ethyloxycarbonyl-4-aminophenol | | 0.5% |
| Water | qs | 100.0% |
| Phase C | | |
| Carbomers | | 0.4% |
| Water | | 14.6% |

This gel may be prepared in a manner which is conventional to those skilled in the art.

### EXAMPLE 5

### Surfactant-Free O/W Emulsion

| Phase A | | |
|---|---|---|
| N-ethyloxycarbonyl-4-aminophenol | | 0.5% |
| Plant oils | | 12.0% |
| UVA screening agent | | 2.0% |
| WB screening agents | | 4.0% |
| Cyclopentasiloxane | | 6.0% |
| Phase B | | |
| Flavosterone SB (Ichimaru Pharcos) | | 2.0% |
| Disodium EDTA | | 0.05% |
| Diglycol/CHDM/isophthalates/SIP copolymer | | 2.0% |
| (Eastman AQ 38S from Eastman Chemical) | | |
| Glycerol | | 5.0% |
| Ethanol | | 10.0% |
| water | qs | 100.0% |

A bleaching cream which is particularly suitable for sensitive or reactive skin is obtained.

This cream may be prepared in a manner which is conventional to a person skilled in the art.

French patent application 0014479 filed Nov. 10, 2000, is incorporated herein by reference.

Where ranges are stated herein all subranges therebetween, and all values therebetween, are incorporated as if written out, and make up a part of the invention.

As noted above, the invention also includes a method of preparing the invention composition, which method comprises mixing together the invention aminophenol derivative and at least one isoflavonoid, where the term “mixing together” includes all forms and orders of additions, contact, etc. such that both components end up in the same composition, regardless whether physically in contact in the composition or solubilized, suspended, etc. in different phases, etc. of the composition.

The invention composition may be used in a conventional manner to depigment and/or bleach the skin, preferably human skin, such as by application thereto once or more per day for one day or several, as needed. Particular areas of application include skin to be depigmented and/or bleached, such as those areas and conditions described in the Background section above. It is within the skill of the ordinary artisan to use the invention compositions for such purposes, for example by applying 0.1-10 g of composition to affected areas of the skin. In like fashion, the invention composition, or its ingredients, can be added to other materials such as base materials (emulsions, waxes, solutions, gels, pastes, etc.) to form a depigmenting and/or bleaching preparation for the skin, especially human skin.

1. A composition comprising at least one aminophenol derivative and at least one isoflavonoid.

2. The composition according to claim 1, wherein the aminophenol derivative is a compound of formula (I): 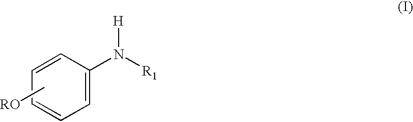
in which:
R represents a hydrogen atom or a radical —COR₂,
where R₂ represents a radical chosen from a saturated or unsaturated, linear, cyclic or branched, optionally hydroxylated C₁-C₃₀ alkyl or alkoxyl radical,
R₁ is selected from the group consisting of radicals of formula (a), (b) or (c):
(a) —CO—NR₃R₄
(b) —CO—O—R₅
1 (c) —SO₂—R₅
where R₃ represents a hydrogen atom or a linear or branched, saturated or unsaturated, optionally hydroxylated C₁-C₆ alkyl radical,
where R₄ represents a hydrogen atom or a radical selected from the group consisting of saturated or unsaturated, linear, cyclic or branched, optionally hydroxylated C₁-C₃₀ alkyl radicals,
where R₅ represents a radical selected from the group consisting of saturated or unsaturated, linear, cyclic or branched, optionally hydroxylated C₁-C₃₀ alkyl radicals.

3. The composition according to claim 2, wherein said aminophenol derivative satisfies at least one of the following conditions:
R represents a hydrogen atom,
the function —OR on the phenyl radical is in an ortho or para position to NHR₁,
R₁ is a radical of formula (a) or (b).

4. The composition according to claim 1, wherein said aminophenol derivative is selected from the group consisting of N-ethyloxycarbonyl-4-aminophenol; N-ethyloxycarbonyl-O-ethyloxycarbonyl-4-aminophenol; N-cholesteryloxycarbonyl-4-5 aminophenol and N-ethylaminocarbonyl-4-aminophenol.

5. The composition according to claim 1, wherein said aminophenol derivative is N-ethyloxycarbonyl-4-aminophenol.

6. The composition according to claim 1, comprising from 0.01 to 10% by weight of the aminophenol derivative relative to the total weight of the composition.

7. The composition according to claim 6, comprising from 0.5% to 3% by weight of aminophenol derivative relative to the total weight of the composition.

8. The composition according to claim 1, comprising from 0.0001 to 10% by weight of isoflavonoid relative to the total weight of the composition.

9. The composition according to claim 8, comprising from 0.001 to 1% by weight of isoflavonoid relative to the total weight of the composition.

10. The composition according to claim 1, wherein said isoflavonoid is an isoflavonoid of natural origin.

11. The composition according to claim 1, wherein said isoflavonoid is selected from the group consisting of daidzin, genistin, daidzein, formononetin, cuneatin, genistein, isoprunetin and prunetin, cajanin, orobol, pratensein, santal, junipegenin A, glycitein, afrormosin, retusin, tectorigenin, irisolidone, jamaicin, and mixtures thereof.

12. The composition according to claim 1, comprising an isoflavone.

13. The composition according to claim 1, further comprising at least one UV screening agent and/or at least one keratolytic agent and/or desquamating agent.

14. The composition according to claim 1, wherein said composition comprises a synergistic combination of said at least one aminophenol derivative and said at least one isoflavonoid.

15. The composition according to claim 3, wherein said aminophenol derivative satisfies all three conditions.

16. The composition according to claim 6, comprising from 0.0001 to 10% by weight of isoflavonoid relative to the total weight of the composition.

17. The composition according to claim 7, comprising from 0.001 to 1% by weight of isoflavonoid relative to the total weight of the composition.

18. A method of bleaching and/or depigmenting the skin, comprising applying the composition of claim 1 to human skin.

19. A method of bleaching and/or depigmenting the skin, comprising applying the composition of claim 17 to human skin.

20. A method for the preparation of a composition, comprising mixing together at least one aminophenol derivative and at least one isoflavonoid.
